(19) United States
(12) Patent Application Publication
(10) Pub. No.: US 2010/0113967 A1
(43) Pub. Date: May 6, 2010

(54) CATHETER WITH PRESSURE SENSOR

(75) Inventor: Donald E. Bobo, SR., Fountain Valley, CA (US)

Correspondence Address: INSKEEP INTELLECTUAL PROPERTY GROUP, INC, 2281 W. 190TH STREET, SUITE 200, TORRANCE, CA, 90504, US

(73) Assignee: InnerSpace, Inc.

(21) Appl. No.: 12/606,160

(22) Filed: Oct. 26, 2009

Related U.S. Application Data
(60) Provisional application No. 61/197,041, filed on Oct. 24, 2008.
(60) Provisional application No. 61/197,039, filed on Oct. 24, 2008.

(51) Int. Cl. A61B 5/00; A61B 1/00

(52) U.S. Cl. 600/561; 600/101

(57) ABSTRACT

A pressure sensor assembly is disclosed which includes a flaccid tube having two mounting sleeve members that bond the tube to a distal end of a catheter. The catheter includes an aperture located beneath the tube and in communication with an air passage. As the pressure outside the tube changes, the tube moves relative to the catheter body, thereby communicating that pressure change to the sealed air passage within the catheter. This air passage is connected to an external transducer that can measure this pressure change and thereby determine a pressure at the distal end of the pressure catheter.

### RELATED APPLICATIONS

This application claims priority to U.S. Provisional Application Ser. No. 61/197,039 filed Oct. 24, 2008 entitled A Single Lumen Catheter with Separate Tubes Therein and U.S. Provisional Application Ser. No. 61/197,041 filed Oct. 24, 2008 entitled A Catheter with an Integrated Pressure Sensor all of which are hereby incorporated herein by reference.

### BACKGROUND OF THE INVENTION

During medical procedures, catheters are often inserted into various locations of a patient, such as vessels, ducts, and body cavities. During many catheter procedures, it can be valuable to sense the pressure within the patient, for example, to determine blood pressure or intracranial pressure. However, the pressure sensing equipment on prior art pressure sensing catheters often make them unsuitable or undesirable for many diagnostic or treatment purposes.

For example, U.S. Pat. No. 4,722,348, the contents of which are hereby incorporated by reference, is directed to a catheter having a pressure transducer in its tip that connects to specialized display equipment. Generally, these catheters are relatively expensive to manufacture due to the integrated transducer and therefore do not always achieve a price point suitable for disposable use. Further, these catheters often require specialized and expensive equipment that connect to this catheter, which further increases the cost of use for such a product.

In another example, such as U.S. Pat. No. 6,447,462, a large sleeve bladder is located on a distal end of a catheter. The bladder is often composed of a material, the proximal and distal ends of which are bonded to the catheter body. This bladder, when inflated, is generally about twice the diameter of the catheter body over which it is located. When placed within a patient, it is largely collapsed after which a small amount of air is added. The bladder of the catheter, once in the body, is therefore folded or furled about the catheter. Prior art catheters effect a volume change by compressing or expanding in a manner that changes the effective circumference of the sensor. In contrast, the circumference of the present invention is constant. A variable volume chamber is formed by placing a flaccid sleeve on the outside of the catheter and passing a small diameter tube through the chamber thus formed. The volume of the chamber is the annular area formed by the larger and smaller diameter times the chamber length. A change in pressure causes the flaccid tube to become more or less elliptical. The change in shape changes the annular area and thus the volume of the chamber. The chamber volume changes as pressure changes in accordance to Boyle's law.

These pressure sensing catheters generally have several shortcomings. First, the bladder typically must have a relatively long length, which prevents much of the distal end of the catheter from being used for other purposes. For example, there is very little space for a desirable number of drainage apertures leading to a drainage lumen.

Second, these shrink bladders have a generally large diameter due to the size of the bladder and its furled configuration. Hence, these catheters are not suitable for smaller diameter uses such as in arterial lines, PICC lines and central venous catheters.

Finally, these shrink bladders are often not suitable for uses that require that the catheter be forcibly pushed through skin and tissue as is the case in a central venous catheter as the bulk of the folded bladder increases the difficulty of insertion and the trauma to the tissue through which it passes.

Therefore, there is a need for an improved pressure sensing catheter that can maintain a relatively small diameter, a relatively short length, lower manufacturing cost and does not add to the difficulty of placing a catheter through skin.

### SUMMARY OF THE INVENTION

A preferred embodiment of the present invention describes a pressure sensor assembly that creates a variable volume chamber by mounting a flaccid tube on a major diameter (i.e., larger diameter) such as two mounting sleeve members that bond the tube to the body of a catheter and a minor diameter (i.e., smaller diameter) provided by a smaller diameter tube that passes within OD of the tube. The catheter includes an aperture located beneath the tube and in communication with an air passage. As the pressure outside the flaccid tube changes, the tube shape becomes more or less elliptical and thereby changes the annular area between the major and minor diameter. Thus the chamber volume changes in response to pressure change according to Boyle's law. An air passage extends from the chamber to the proximal end of the catheter where it is connected to an external transducer that can measure the pressure sensed by the bladder

In another preferred embodiment, the catheter body beneath the tube can have a “necked” or reduced diameter relative to the adjacent areas of the catheter body. This allows the pressure sensor assembly to the same outer diameter as the outer diameter of the catheter body.

In another preferred embodiment, the pressure sensor assembly can be covered by a sheath having a plurality of apertures or slits. This sheath provides physical protection to the balloon while allowing pressure from the patient's body to be communicated to the tube.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 illustrates a side cross sectional view of a pressure sensor assembly according to a preferred embodiment of the present invention;

FIGS. 2A and 2B illustrate various views of a mounting sleeve member of the embodiment of FIG. 1;

FIGS. 3A and 3B illustrates various views of a flaccid tube of the embodiment of FIG. 1;

FIG. 4 illustrates a side cross sectional view of the flaccid tube and mounting sleeve members of FIG. 1;

FIG. 5 illustrates an exploded cross sectional view of the flaccid tube and mounting sleeve member of FIG. 1;

FIG. 6 illustrates a side cross sectional view of a catheter body according to a preferred embodiment of the present invention;

FIG. 7 illustrates a side cross sectional view of a pressure sensor assembly according to a preferred embodiment of the present invention;

FIGS. 8A and 8B illustrates various views of an apertured sheath according to a preferred embodiment of the present invention;

FIG. 9 illustrates the sheath of FIGS. 8A and 8B covering a pressure sensor assembly according to a preferred embodiment of the present invention;

FIGS. 10A and 10B illustrates various views of a sheath with fingers according to a preferred embodiment of the present invention;

FIG. 11 illustrates the sheath of FIGS. 10A and 10B according to a preferred embodiment of the present invention;

FIG. 12 illustrates a side cross sectional view of a pressure sensing assembly according to a preferred embodiment of the present invention

FIG. 13 illustrates a side view of a pressure sensing catheter with a distal drainage area according to the present invention; and,

FIG. 14 illustrates a top view of a pressure sensing catheter with an offset distal passage according to the present invention;

FIG. 15 illustrates a cross sectional side view along lines A-A of FIG. 14; and,

FIG. 16 illustrates a cross sectional view along lines B-B of FIG. 14.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

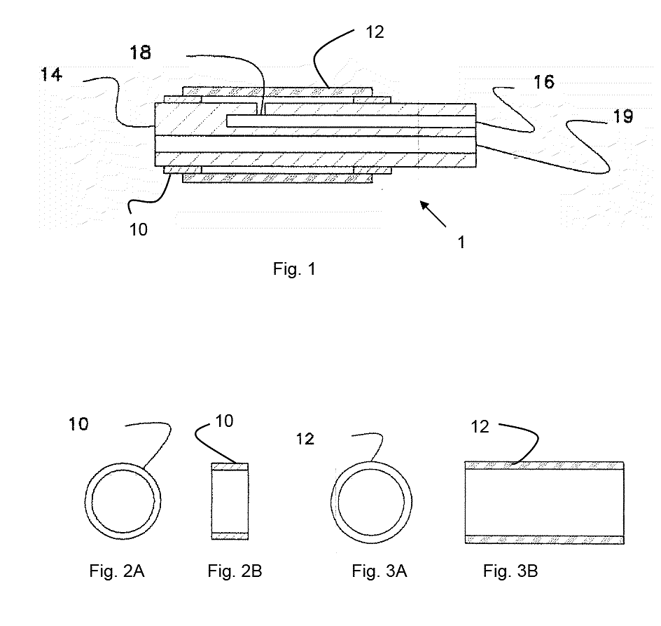

FIG. 1 illustrates a distal end of a catheter having a pressure sensing assembly 1 according to a preferred embodiment of the present invention. The pressure sensor assembly 1 provides pressure sensing functionality without significantly increasing the overall diameter of the catheter, as seen in prior art catheters. Hence, the pressure sensor assembly 1 can be used for many different purposes that larger prior art catheters could not, such as in arterial lines, in PICC lines and central venous catheters. Further, the design of the present invention does not require any pressure sensing electronics to be located in the catheter itself, allowing catheters of this embodiment to be lower cost and “disposable” as compared with more expensive and reusable prior art designs. Finally, the length of the pressure sensor assembly 1 is much shorter than the prior art heat shrink bladder pressure sensors which allows more room on the distal end of the catheter for other features or functionality.

The pressure sensing assembly is preferably composed of a catheter body 14 having an air lumen passage 16 that extends along most of the length of the catheter body 14. An aperture 18 connects to and is in communication with the air lumen passage 16 near a distal end of the passage 16. The aperture 18 is in communication with a space formed by a flexible or flaccid tube 12 (also seen in FIGS. 3A and 3B) and two mounting sleeve members 10 (also seen in FIGS. 2A and 2B).

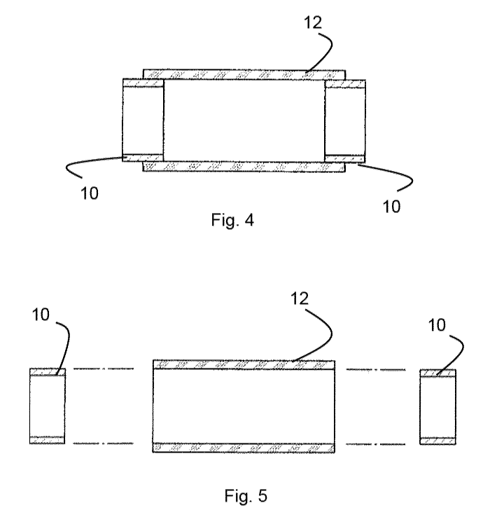

As seen in FIGS. 4 and 5, an outer surface of each mounting sleeve members 10 are bonded to an inner surface of the flaccid tube 12 in an airtight manner. Preferably, the mounting sleeve members 10 are composed of a polymer and are preferably bonded with a UV cured adhesive. The flaccid tube 12 is preferably composed of butyl, preferably has a thickness of about 0.003 inches, preferably has a length (defined by the major and minor diameters of the catheter that is between about 0.03 and 0.60 inches, and preferably has a hydrophilic coating.

Preferably, the one mounting sleeve member 10 is first bonded to a desired location adjacent the aperture 18 on the distal end of the catheter body 14. Next, the other mounting sleeve member 10 is moved towards the first mounted sleeve member 10, thereby creating some slack in the flaccid tube 12. When a desired amount of slack between the proximal and distal ends of the tube 12 has been created (i.e., longitudinal slack), the second mounted sleeve member 10 is also bonded to the catheter body 14. Preferably, enough slack is introduced into the flaccid tube 12 that it can move or deflect to a generally elliptical shape, and more specifically enough to provide a V1/V2 ratio to allow measurement of pressure from 720 mm Hg to 1060 mm Hg. for a given length variable volume chamber.

As previously discussed, the air lumen passage 16 extends through the length of the catheter. Preferably, the air lumen passage 16 opens near the proximal end of the catheter in a manner connectable with additional pressure sensing equipment, such as a pressure transducer. Hence, when the pressure sensing apparatus 1 is positioned within a patient, the pressure around the catheter applies pressure on the flaccid tube 12. The flaccid tube 12 presses on the gas (e.g., air) or liquid (e.g., saline) underneath it, changing the pressure within the air lumen passage 16 which can be ultimately measured via a connected transducer or similar system.

Finally, the catheter body 14 can also be used for other functionality via a through lumen passage 19. For example, this passage can be used for a guidewire, fluid delivery or drainage (discussed further below with regard to FIG. 4).

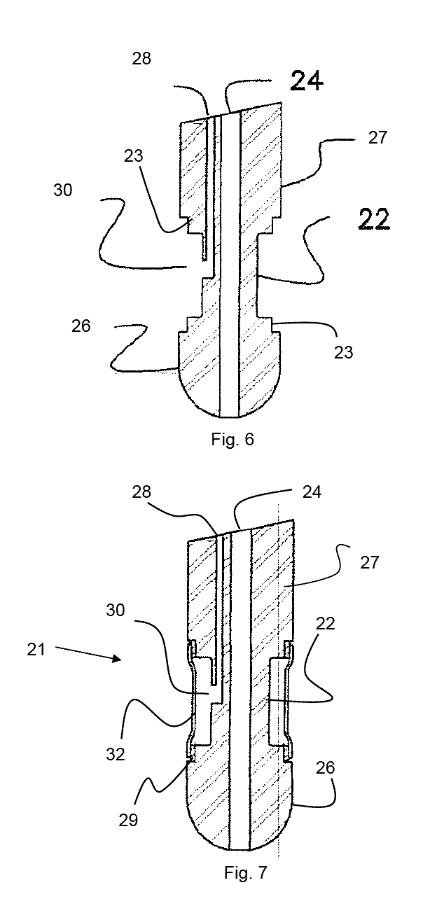

FIGS. 6 and 7 illustrate another preferred embodiment according to the present invention of a pressure sensing assembly 21 on a distal end of a catheter body 27. More specifically, the assembly 21 is arrange such that the diameter profile of the distal end of the catheter body 27 is uniform or even smaller as compared with the diameter profile of other areas (e.g., mid and proximal areas) of the catheter body 27. In other words, the area near the pressure sensing assembly 21 is relatively uniform or smaller in diameter than adjacent areas of the catheter body 27. This embodiment may be especially desirable for uses that require a substantial amount of flexibility and a uniform catheter diameter, such as with a central venous catheter (CVC) or a peripherally inserted central catheter line (PICC line).

As with the previously described assembly 1, the assembly 21 includes a through lumen 24 and an air lumen passage 28. A distal end of the air lumen passage 28 opens to an aperture 30 at a “necked” or recessed area 22. The recessed area 22 has a smaller diameter than nearby portions of the catheter body 27.

The proximal and distal ends of the recessed area 22 are located adjacent second recessed areas 23. The second recessed areas 23 are preferably recessed to a depth to accommodate the mounting sleeve members 29 and flaccid tube 32. Preferably, when the mounting sleeve members 29 and flaccid tube 32 are mounted on the second recessed areas 23, the diameter of these areas is preferably about flush with the adjacent areas of the catheter body 27 less about twice the thickness of the flaccid tube 32.

In this respect, the pressure sensing assembly 21 transmits or communicates pressure outside the catheter body 27 (e.g., from a lumen of a patient) to the inside of the catheter body 27 (e.g., within the air lumen passage 28 and to a measuring device such as a transducer).

Finally, it should be noted that the distal end 26 of the catheter body 27 is generally rounded so as to prevent trauma to the patient during use.

In some circumstances, it may be desirable to protect the flaccid tube 32 from damage during use. For example, a central venous catheter in a blood vessel may require that the catheter be forcibly pushed through skin and tissue.

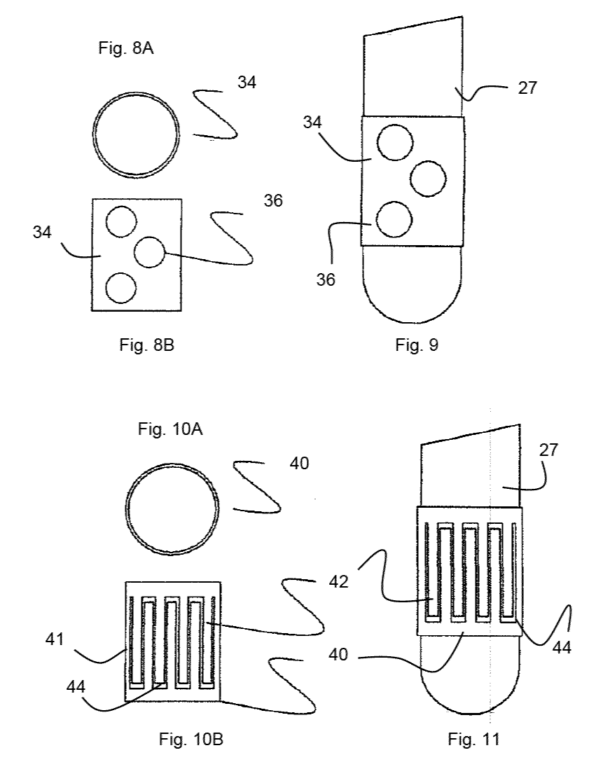

FIGS. 8A and 8B illustrate a protective sheath 34 that can be fixed over the flaccid tube 32 (or 12 from the embodiment of FIG. 1) for protection purposes. Preferably, this sheath 34 is composed of a relatively rigid, inelastic polymer with a relatively thin thickness. Preferably, the sheath 34 has a thickness of about 0.002 inches. Preferably, the sheath 34 is placed over the flaccid tube 32 and its proximal and distal ends are bonded to the catheter body 27. Alternately, the sheath may be a continuous portion of the catheter body 27.

Additionally, the sheath 34 includes a plurality of apertures 26 that allow communication of pressure from the outside environment to the flaccid tube 32 and therefore to the air lumen passage 28. Preferably, the apertures 36 have a diameter that ranges between about 0.02 and 0.04. Hence, the sheath 34 protects the flaccid tube 32 from damage (e.g., such as insertion stress) while avoiding interference with the movement and pressure communicating functionality of the flaccid tube 32.

FIGS. 10A, 10B and 11 illustrate various views of an alternate preferred embodiment of a sheath 40 that can be bonded over the flaccid tube 32. The sheath 40 includes a gap 44 having an undulating pattern around a portion of the sheath 40 so as to create finger regions 42. Preferably, the gap 44 does not continuously extend around the entire circumference of the sheath 40, allowing connecting regions 41 to connect the proximal and distal ends of the sheath together.

Preferably, the fingers 42 are bonded together with a water-soluble adhesive, either along portions of the gap 44 or along the entire gap 44. Since this adhesive is water-soluble, it will maintain the relative position of the fingers 42 prior to advancing the catheter into a patient and for a period of time within the patient. However, after a predetermined period of time in the patient, the adhesive will degrade, allowing the fingers 42 to move freely.

The shape of the strips or fingers 42 allow them to move independently from one another (after any adhesive has degraded) and, for example, resist the drag on the patient's skin as the catheter is advance or retracted. Since catheters that are located within a body for longer periods can build up protein and hence clog small apertures or adhere different components together, the flexibility of the fingers 42 of the sheath 40 may reduce interference of this protein build up by retaining flexibility.

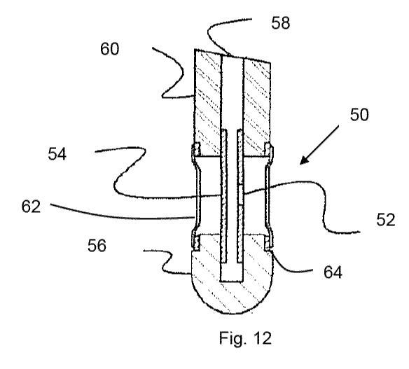

FIG. 12 illustrates another preferred embodiment of a pressure sensing assembly 50 according to the present invention. More specifically, the assembly 50 is preferably composed of a catheter body 60 having an air passage 58 and a rounded nose member 56 at a distal end of the assembly 60 that are bonded together by a tube 54. The tube 54 includes an internal passage in communication with the air passage 58. An aperture 52 in the tube 54 allows the passage within the tube 54 to communicate with a space surrounded by a flaccid tube 62 and mounting sleeve members 64.

By using multiple components to compose the assembly 50, the space between the catheter body 60 and the curved nose member 56 (and therefore the amount of slack in the flaccid tube 62) can be more easily adjusted during assembly. Additionally, the use of the tube 54, which has a relatively small diameter, may allow for the overall catheter diameter to be further reduced.

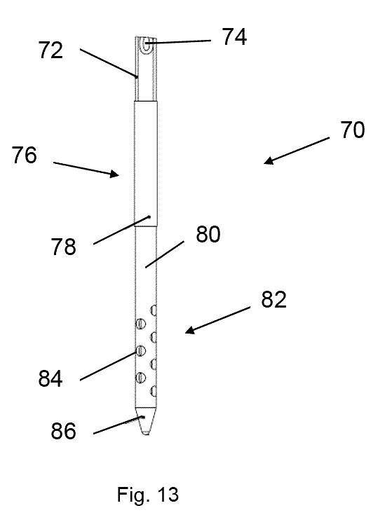

FIG. 13 illustrates an example catheter 70 having a pressure sensing assembly 76 with a flaccid tube 78 disposed over a portion of the catheter body 72 as described in any of the previous embodiments. Additionally, the distal end of the catheter 76 includes a drainage assembly 82 having a cylindrical member 80 with an internal passage that connects to a through lumen (such as through lumen 24 in FIG. 7). Apertures 84 in the cylindrical member 80 allow the passage in the member 80, and therefore the through lumen of the catheter 70, to communicate with the outside environment and hence be used for drainage. A soft dip 86 is located at the end of the cylindrical member 80 so as to reduce contact-related trauma. Finally, the catheter body may also include another internal passage that terminates at aperture 74, allowing the user to deliver fluids or other treatments to a location within the patient.

### Example 1

A variable volume sensor assembly suitable for use in a catheter is formed by placing a set of sleeves on either side of an aperture passing through a wall of the catheter and into an internal lumen that leads to an external pressure transducer. Each end of a flaccid tube is bonded to one of the sleeves, forming an annulus defined by the internal diameter of the sleeve and the outer diameter of the catheter body beneath the sleeve. The volume of the enclosed space can be determined by multiplying the area of the annulus times the distance between the sleeves.

During use, the flaccid tube changes shape in response to changes in pressure, which therefore changes the area of the annulus. This area change is analogous to the change in area that occurs when a circle is deformed to the shape of an ellipse. The circumference of the circle and the ellipse is the same, but the area is different. As the area of the tube changes, the area of the annulus that defines the volume of the sensor changes and hence the volume of the sensor thereby changes with pressure.

The reaction of the flaccid tube to pressure thereby forms a variable volume pressure sensor that operates in accordance with Boyle's law. A preferred aspect in the manufacture of the sensor is to have the flaccid tube be slightly longer than the length of the minor diameter segment. The slack that is created allows the tube to change shape without being stretched. This slack therefore allows the tube to change shape without being constrained by tensile forces.

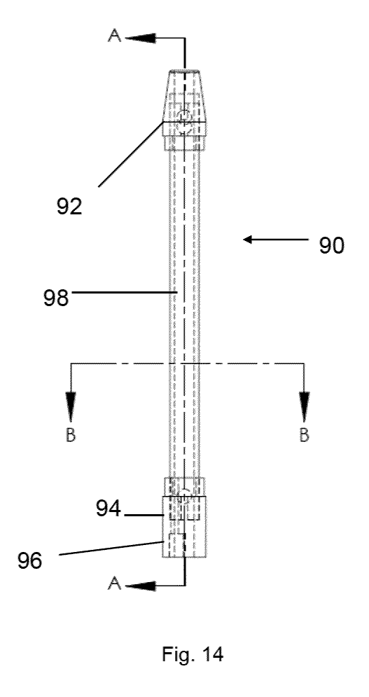

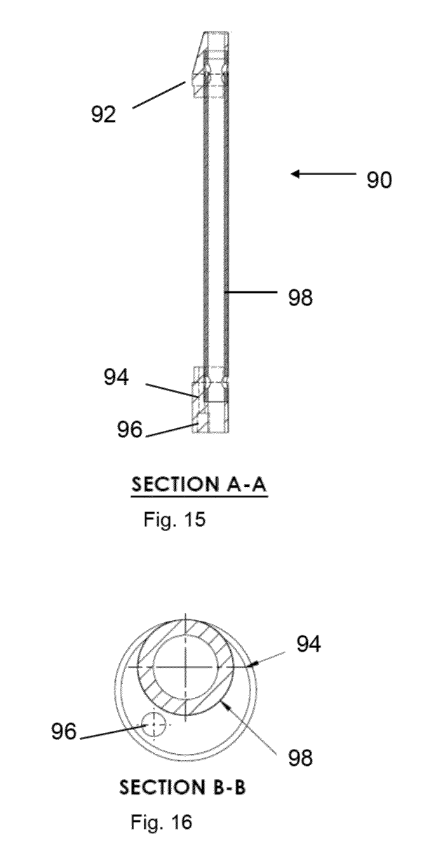

FIGS. 14-16 illustrate another embodiment of a pressure sensing assembly 90 use for pressure sensing (shown here without a flaccid tube for purposes of clarity). The assembly 90 is generally similar to that of assembly 50 in FIG. 12. For example, body 96 and tip 92 provide a larger diameter with recessed areas for bonding a flaccid tube. A smaller diameter tube 98 connects the body 96 with the tip 92 and provides a pass through lumen for other purposes. An air lumen 96 terminates at the end of the body 94 so as to communicate with the area beneath the flaccid tube. However, as best see in FIG. 16, the tube 98 is offset from the center of the body 94. This arrangement provides a different shaped area beneath the flaccid tube that, for some uses, may provide more accurate measurement.

It should be understood that while different embodiments have been discussed as using air within the catheter for communicating pressure measurement, other gasses and fluids may also be used.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A pressure sensing catheter comprising:
a catheter body having a first passage extending from near a proximal end of said catheter body to a location on said catheter body distal to said proximal end; said first passage having a proximal end connectable to a pressure transducer;
a first mounting sleeve member disposed on said catheter body;
a second mounting sleeve member disposed on said catheter body;
a flaccid tube disposed over a portion of said catheter body to have longitudinal slack, said flaccid tube having a proximal end and a distal end forming an airtight connection;
an aperture located through said catheter body between said first passage and a space formed underneath said tube.

2. The pressure sensing catheter of claim 1, wherein said flaccid tube is mounted to said catheter body such that said flaccid tube can deflect enough to provide a V1/V2 ratio to allow measurement of pressure from 720 mm Hg to 1060 mm Hg. for a given length variable volume chamber.

3. The pressure sensing catheter of claim 1, wherein said catheter body comprises a first area of reduced diameter and a second area of reduced diameter; wherein said first mounting sleeve is disposed on said first area of reduced diameter and said second mounting sleeve is disposed on said second area of reduced diameter.

4. The pressure sensing catheter of claim 3, wherein said first area of reduced diameter and said second area of reduced diameter are offset from an adjacent portion of said catheter body such that an outer diameter of said flaccid tube is about even with and outer diameter of said adjacent portion of said catheter body.

5. The pressure sensing catheter of claim 4, further comprising a third area of reduced diameter disposed between said first area of reduced diameter and said second area of reduced diameter and being smaller in diameter than said first area of reduced diameter and said second area of reduced diameter.

6. The pressure sensing catheter of claim 5, wherein said third area of reduced diameter is a tube.

7. The pressure sensing catheter of claim 1, further comprising a rigid sheath fixed from movement over said flaccid tube.

8. The pressure sensing catheter of claim 7, wherein said sheath further comprises a plurality of apertures.

9. The pressure sensing catheter of claim 7, wherein said sheath further comprises a plurality of gaps.

10. A catheter for sensing pressure within a patient comprising:
an elongated catheter body having a first passage extending from a proximal end of said catheter along a length of said catheter; said first passage being connectable to an external pressure transducer;
an aperture disposed near a distal end of said catheter body and passing from an outside of said catheter body to said first passage;
a flaccid tube located over a portion of said catheter and said aperture; a proximal and distal end of said flaccid tube sealingly bonded to said catheter body so as to form a variable volume chamber.

11. The catheter of claim 10, wherein said flaccid tube is mounted relative to said catheter body so as to deflect enough to provide a V1/V2 ratio to allow measurement of pressure from 720 mm Hg to 1060 mm Hg. for a given length variable volume chamber.

12. The catheter of claim 10, wherein said catheter body further comprises a first area having a reduced diameter; said first area being located beneath said flaccid tube.

13. The catheter of claim 12, wherein said first area comprises a tube bonded to a distal tip of said catheter body and a proximal body portion of said catheter body.

14. The catheter of claim 12, wherein said first area further comprises a distal step located at a distal end of said first area and a proximal step located at a proximal end of said first area; said flaccid tube bonded to said proximal step and said distal step.

15. The catheter of claim 14, further comprising a proximal mounting sleeve located between said flaccid tube and said proximal step and a distal mounting sleeve located between said flaccid tube and said distal step.

16. The catheter of claim 15, further comprising a sheath disposed over said flaccid tube.

17. The catheter of claim 16, wherein said sheath further comprises a plurality of openings through said sheath.

18. A catheter for sensing pressure within a patient comprising:
an elongated catheter body having an air passage extending between a proximal end of said catheter body and a distal end of said catheter body;
a proximal end of said air passage being connectable to an external pressure transducer;
said catheter body having a reduced diameter region near said distal end of said catheter body;
an aperture located through a portion of said catheter body so as to allow communication between said air passage and an environment outside of said catheter body;
a flaccid tube bonded over said reduced diameter region and said aperture; said flaccid tube being positioned to include longitudinal slack in said flaccid tube so as to allow deflection of said flaccid tube into and out of an area around said reduced diameter region.

19. The catheter for sensing pressure of claim 18, further comprising a rigid tubular member disposed over said flaccid tube.

20. The catheter for sensing pressure of claim 19, wherein said sheath includes a plurality of openings through a wall of said sheath.
